(19) United States
(12) Patent Application Publication
(10) Pub. No.: US 2026/0263690 A1
(43) Pub. Date: Sep. 10, 2026

(54) INTRAVENOUS BLOOD SET FOOT PUMP

(71) Applicant: CAREFUSION 303, INC, San Diego, CA (US)

(72) Inventors: Vincent Vinh NGUYEN, Placentia, CA (US); Rahul Rajan PUTHUKKAD, Bengaluru (IN); Abhishek KRISHNAN, Bengaluru (IN); Sumit RAJPAL, Panipat (IN); Kaushik SUMAN, Jharkhand (IN); Srinivas Ramakrishnarao PEDDI, Pune (IN); Aman DESAI, Bengaluru (IN)

(21) Appl. No.: 19/660,705

(22) Filed: Apr. 28, 2026

Related U.S. Application Data
(62) Division of application No. 17/984,089, filed on Nov. 9, 2022.
(60) Provisional application No. 63/282,408, filed on Nov. 23, 2021.

(51) Int. Cl. A61M 5/142; A61M 5/152; A61M 5/168

(52) U.S. Cl. CPC A61M 5/1424; A61M 5/152; A61M 5/16881

(57) ABSTRACT

An intravenous (IV) blood set foot pump including a housing comprising a fluid cavity, an inlet port, and an outlet port, a squeezing member coupled to the housing, and a biasing member coupled to the housing and the squeezing member, the biasing member configured to exert a biasing force against the squeezing member when the biasing member is in a compressed state, wherein the inlet port is configured to be coupled to a first IV tube of an IV set and the outlet port is configured to be coupled to a second IV tube of the IV set. IV sets with IV blood set foot pumps and methods of operating IV blood set foot pumps are also provided.

### CROSS-REFERENCE TO RELATED APPLICATIONS

This application is a divisional of U.S. patent application Ser. No. 17/984,089, entitled “INTRAVENOUS BLOOD SET FOOT PUMP,” filed on Nov. 9, 2022, which claims the benefit of priority under 35 U.S.C. § 119 to U.S. Provisional Patent Application Ser. No. 63/282,408, entitled “INTRAVENOUS BLOOD SET FOOT PUMP,” filed on Nov. 23, 2021, the entirety of which is incorporated herein by reference.

### TECHNICAL FIELD

The present disclosure generally relates to intravenous (IV) sets, in particular to foot pumps for IV blood sets.

### BACKGROUND

Medical treatments often include the infusion of a medical fluid (e.g., blood, plasma) to patients using an IV catheter that is connected though an arrangement of flexible tubing and fittings, commonly referred to as an “IV set,” to a source of fluid, for example, an IV blood bag. During operation, medical fluid may be required quickly at greatly increased flow rates as shorter times to blood transfusions have been associated with decreased death risk in trauma patients. Typical IV blood sets use a manual hand pump bulb or flexible container that is squeezed by hand to rapidly increase fluid flow rate, resulting in muscle fatigue.

For these reasons, it is desirable to provide an IV blood set pump that is foot operated to eliminate hand muscle fatigue and to free up the user's hands.

### SUMMARY

In one or more embodiments, an intravenous (IV) blood set foot pump including a housing comprising a fluid cavity, an inlet port, and an outlet port, a squeezing member coupled to the housing, and a biasing member coupled to the housing and the squeezing member, the biasing member configured to exert a biasing force against the squeezing member when the biasing member is in a compressed state, wherein the inlet port is configured to be coupled to a first IV tube of an IV set and the outlet port is configured to be coupled to a second IV tube of the IV set.

In one or more embodiments, an intravenous (IV) blood set foot pump comprising a frame configured to be removably coupled to an IV pole, the frame comprising an air inlet port, a piston slot, and a pump slot configured to receive a diaphragm pump segment of an IV set, a piston moveably coupled to the frame through the piston slot, an air hose, wherein a first end of the air hose is coupled to the air inlet port of the frame, and a foot pump coupled to a second end of the air hose.

The foregoing and other features, aspects and advantages of the disclosed embodiments will become more apparent from the following detailed description and accompanying drawings.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIG. 2 depicts a top view of a typical assembled IV infusion set with a hand pump.

FIG. 3 depicts a front view of an IV blood set foot pump, according to aspects of the disclosure.

FIG. 4 depicts a perspective view of an IV blood set with a foot pump, according to aspects of the disclosure.

FIG. 5 depicts a perspective view of an IV blood set foot pump connecting an infusion pump to a patient, according to aspects of the disclosure.

FIG. 6 depicts a perspective view of an IV blood set foot pump, according to aspects of the disclosure.

FIG. 7 depicts a perspective exploded view of the IV blood set foot pump of FIG. 6, according to aspects of the disclosure.

FIG. 8 depicts a perspective view of an IV blood for use with a foot pump, according to aspects of the disclosure.

FIG. 9 depicts a perspective view of an IV blood set with a foot pump, according to aspects of the disclosure.

FIG. 10 depicts a perspective view of an IV set with an IV set foot pump and an IV pole, according to aspects of the disclosure.

FIG. 11 depicts a perspective view of an IV set foot pump on an IV pole, according to aspects of the disclosure.

FIG. 12 depicts an exploded perspective view of the IV set foot pump of FIG. 11, according to aspects of the disclosure.

FIG. 13 depicts a cross-sectional perspective view of a frame of an IV set foot pump, according to aspects of the disclosure.

FIG. 14 depicts a front view of a piston of an IV set foot pump, according to aspects of the disclosure.

FIG. 15 depicts a front view of a diaphragm pump segment of an IV set, according to aspects of the disclosure.

FIG. 16 depicts a perspective view of an IV set foot pump on an IV pole, according to aspects of the disclosure.

FIG. 17 depicts a perspective view of a diaphragm pump segment of an IV set uncoupled to a piston of the IV set foot pump of FIG. 16, according to aspects of the disclosure.

FIG. 18 depicts a perspective view of the diaphragm pump segment of the IV set coupled to the piston of the IV set foot pump of FIG. 16, according to aspects of the disclosure.

FIG. 19 is a diagram of an equation for design of a diaphragm pump segment, according to aspects of the disclosure.

FIG. 20 is a diagram of an example use of the equation of FIG. 19, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

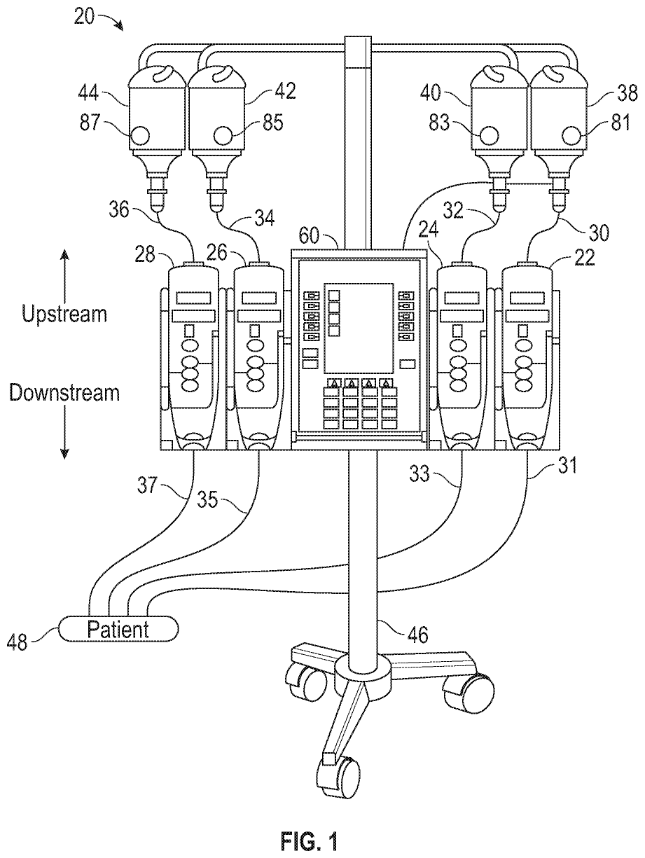

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

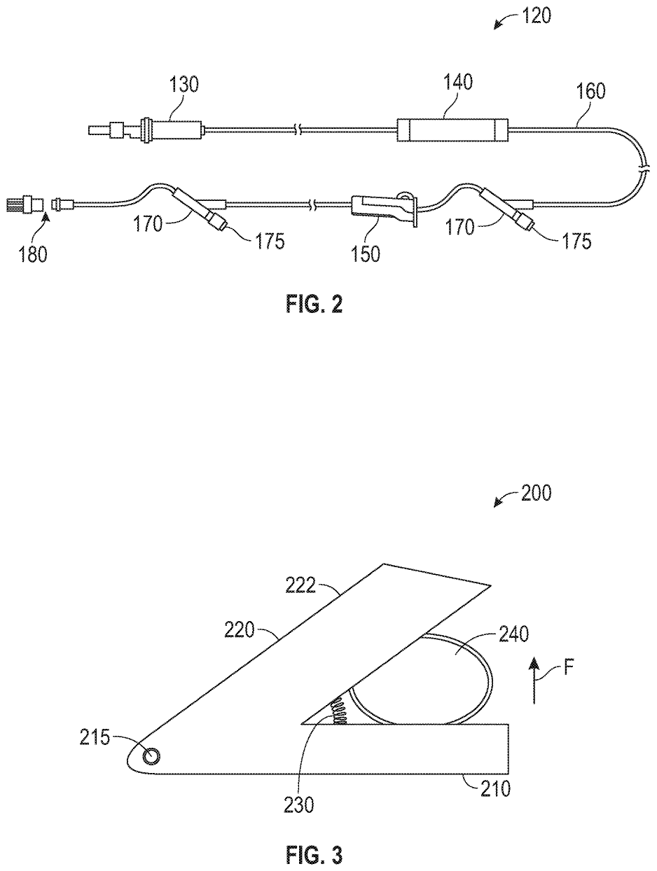

Typically, medical fluid administration sets have more parts than are shown in FIG. 1, such as those shown in FIG. 2. IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2, an IV set 120 may include a drip chamber 130, a hand pump 140 and a roller clamp 150 connected together by tubing 160. The IV set 120 may also include a Y-site 170 having a Y-shaped junction with a needleless connector 175, as well as a luer lock connector 180 at the end of the IV set 120. The luer lock connector 180 may be used for connection to a catheter inserted into a patient, for example. The IV set 120 may include additional infusion components and may be formed of any combination of components and the tubing 160.

In use, IV set 120 is connected to an IV blood bag via the drip chamber 130 and the luer lock connector 180 is connected to a catheter that is placed into a vein of a patient. Thus, blood flows from the blood bag through the drip chamber 130 to the hand pump 140 and through the remainder of the IV set 120 and out of the luer lock connector 180. As the hand pump 140 is squeezed, the volume of blood contained within a body 142 of the hand pump 140 is forced out an outlet check valve 144 and downstream through the IV set 120. When the hand pump 140 is released (e.g., stop squeezing), the body 142 of the hand pump 140 reinflates and draws in a new volume of blood through an inlet check valve 146. This squeezing cycle can be repeated as often as necessary to move blood from one or more blood bags into the patient as quickly as possible.

In aspects of the disclosure, a hand pump may be replaced by a foot pump. Muscles in the leg fatigue less quickly than those in the hand, so the user (e.g., clinician) can pump blood at a higher rate for a longer period of time using a foot pump instead of a hand pump. This also eliminates hand fatigue from pumping blood and frees up the user's hands for other operations.

In aspects of the disclosure, foot pump may be mounted on a base of a bed, mounted on a base of an operating table, mounted on a base of an IV pole or set on a floor surface, for example. The foot pump may be configured to receive a standard blood set hand pump bulb to pump in the blood without hand fatigue. The pumping frequency and corresponding flow rate may be chosen by the clinician based on how quickly and how strongly the foot pump is operated. Here, the hand pump bulb may be inserted into the foot pump so that hand pump bulb is positioned between foot pump surfaces that then squeeze the hand pump bulb to cause the pumping action.

As shown in FIG. 3, an IV blood set foot pump 200 is provided according to aspects of the disclosure. IV blood set foot pump 200 may be in the form of a clamshell design including a base plate 210, a squeezing plate 220 and a biasing member 230. The base plate 210 and the squeezing plate 220 may be moveably coupled by a pivot member 215 (e.g., flexible hinge, hinge pin). The base plate 210 and/or the squeezing plate 220 may have a cavity or indentation configured to receive a standard hand pump bulb or cylinder.

The biasing member 230 may be a spring, a flexible hinge, a compression chamber or any other suitable biasing device. As shown in FIG. 3, the biasing member 230 is a compression spring coupled to both the base plate 210 and the squeezing plate 220. Thus, the biasing member 230 is compressed when the squeezing plate 220 is forced down toward the base plate 210 by a user (e.g., clinician) stepping on a top surface 222 of the squeezing plate 220. The compressed biasing member 230 exerts a biasing force F against the squeezing plate 220. Here, when user pressure on the squeezing plate 220 is released (e.g., user removes or relaxes foot), the biasing force F causes the squeezing plate 220 to move away from the base plate 210 by pivoting at the pivot member 215.

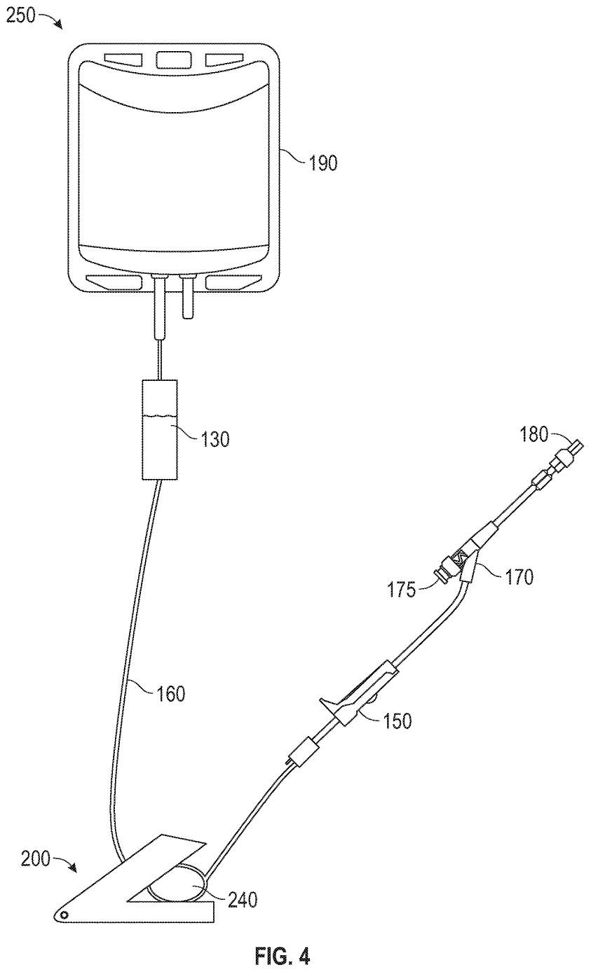

As shown in FIG. 4, the IV blood set foot pump 200 may be part of an IV blood set 250. The IV blood set 250 may include a blood bag 190, a drip chamber 130, a pump bulb 240, a roller clamp 150, a Y-junction 170 having a needleless port 175 and a luer connector 180, all coupled together by IV tubing 160.

In aspects of the disclosure, the IV blood set foot pump 200 is a separate device that receives the pump bulb 240 when necessary. For example, the IV blood set 250 may be in use during a patient procedure as a gravity set or an infusion pump set, then if an issue arises where the blood flow needs to be dramatically increased, the pump bulb 240 may be inserted or placed into the IV blood set foot pump and a clinician can start pumping the IV blood set foot pump 200 by foot. Here, the IV blood set 250 only needs to be coupled to the IV blood set foot pump 200 when needed. As another example, the pump bulb 240 may be inserted into IV blood set foot pump 200 when the IV blood set 250 is put into service, thus providing for immediate use of the IV blood set foot pump when needed.

In aspects of the disclosure, the IV blood set foot pump 200 may be a reusable device. For example, since the IV blood set foot pump 200 is not in direct contact with the blood flowing through the IV blood set 250, the pump bulb 240 may be removed from the IV blood set foot pump 200 and a pump bulb 240 of a replacement IV blood set 250 inserted. Accordingly, IV blood set foot pump 200 may be used for multiple procedures or IV blood set changes between cleanings, or the IV blood set foot pump 200 may be cleaned after every use as dictated by health codes and medical device cleaning protocols.

In use, a bulb (e.g., pump bulb 240) of an IV set (e.g., IV set 250) is inserted or placed into the IV blood set foot pump 200, the pump bulb 240 being positioned between the base plate 210 and the squeezing plate 220. The IV set 250 is coupled to a fluid container (e.g., fluid bag 190) containing a medical fluid (e.g., blood). In an unactuated state, IV blood set foot pump 200 may not compress the pump bulb 240, thus allowing the blood to flow through the IV set 250 at a rate set by a flow controller (e.g., roller clamp 150). When quicker fluid flow is needed, the IV blood set foot pump 200 may be compressed by foot, thus squeezing the fluid out of the pump bulb 240 quickly.

In aspects of the disclosure, the IV blood set foot pump 200 may be a disposable component of the IV blood set 250. For example, the IV blood set foot pump 200 may include an integral or coupled pump bulb 240. Here, the pump bulb 240 of the IV blood set foot pump 200 may be coupled in line via IV tubing 160 to the IV blood set 250. Accordingly, the disposable IV blood set foot pump 200 would be used for the life of the IV blood set only (e.g., 24 hours, 72 hours, 7 days), whereafter the IV blood set foot pump 200 would be disposed of along with the associated IV blood set 250.

In use, the IV set 250 with the disposable IV blood set foot pump 200 is coupled to a fluid container (e.g., fluid bag 190) containing a medical fluid (e.g., blood). In an unactuated state, IV blood set foot pump 200 may not compress the integral/coupled pump bulb 240, thus allowing the blood to flow through the IV set 250 at a rate set by a flow controller (e.g., roller clamp 150). When quicker fluid flow is needed, the IV blood set foot pump 200 may be compressed by foot, thus squeezing the fluid out of the integral/coupled pump bulb 240 quickly.

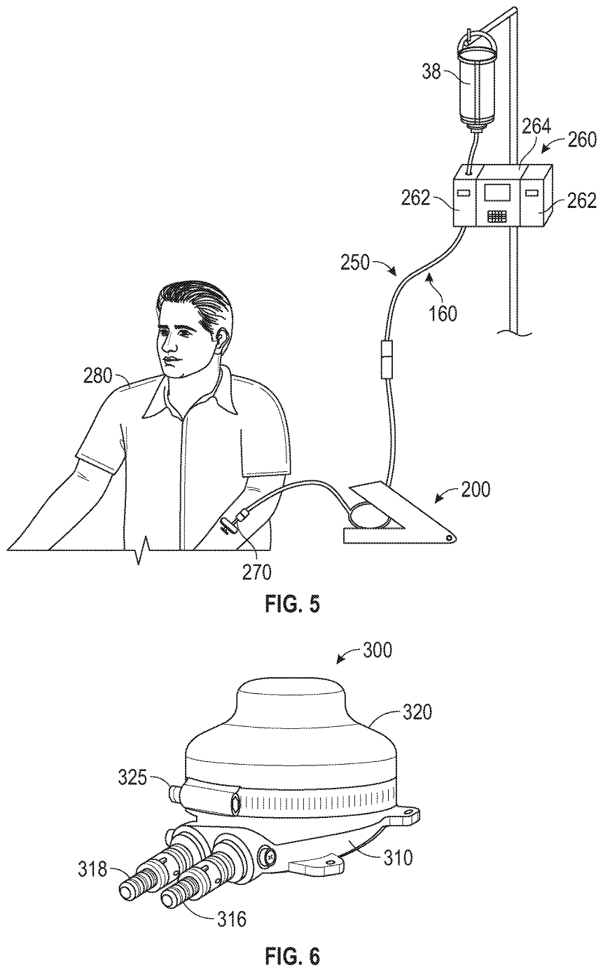

As shown in FIG. 5, the IV blood set 250 and IV blood set foot pump 200 may be coupled to a fluid source 38, to an infusion pump 260 system having two infusion pumps 262 and a controller 264, and to a catheter 270 inserted into a patient 280. Here, if the maximum fluid flow rate from the infusion pump system 260 is not sufficient, the IV blood set foot pump 200 may be activated (e.g., stepped on) to force the fluid to flow more quickly. For example, the IV blood set 250 may be quickly uncoupled from the infusion pump system 260 before using the IV blood set foot pump 200 and/or the infusion pump system 260 may be set to neutral to allow unimpeded fluid flow from the fluid source 38 before using the IV blood set foot pump 200.

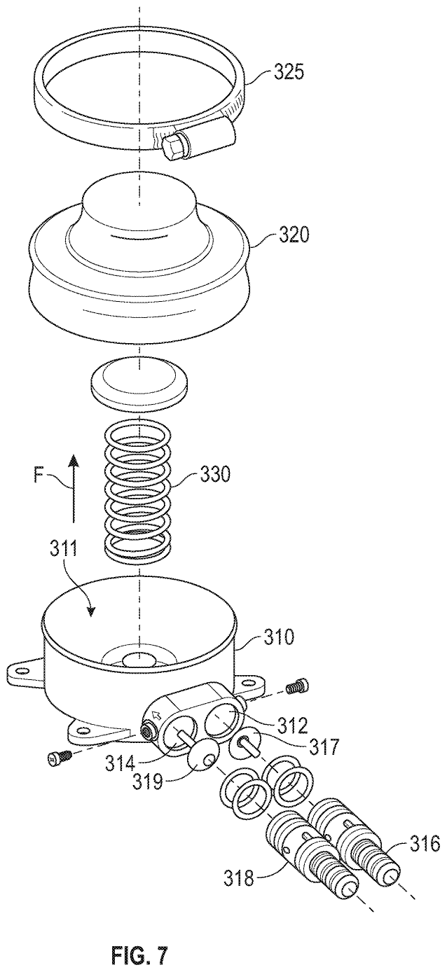

As shown in FIGS. 6 and 7, an IV blood set foot pump 300 is provided according to aspects of the disclosure. IV blood set foot pump 300 may include a housing 310, a squeezing member 320 (e.g., diaphragm) coupled to the housing 310 and a biasing member 330 (e.g. spring) coupled to the housing 310 and the squeezing member 320. The squeezing member 320 may be secured to the housing 310 via a securing member 325. The housing 310 may include an inlet port 312 and an outlet port 314. An inlet connector 316 may be coupled to the inlet port 312 and may include a check valve 317 configured to only allow fluid flow into the housing 310, while an outlet connector 318 may be coupled to the outlet port 314 and may include a check valve 319 configured to only allow fluid flow out of the housing 310.

The squeezing member 320 may be flexible and configured such that pushing inward on the squeezing member 320 increases the pressure (e.g., air pressure, fluid pressure) within a cavity 311 defined by the housing 310 and the squeezing member 320. The increase pressure within the cavity 311 may exceed the cracking pressure of the outlet check valve 319, forcing fluid within the cavity 311 to quickly flow out of the outlet connector 318, while the inlet check valve 317 prevents fluid from flowing out the inlet connector 316. When the squeezing member 320 is released, a biasing force F of the biasing member 330 causes the squeezing member 320 to expand back to an un-compressed state and causing a suction pressure inward towards the cavity 311. Here, the suction pressure may exceed the cracking pressure of the inlet check valve 317 and cause fluid to quickly flow into the cavity 311 through the inlet connector 316, while the outlet check valve 319 prevents fluid from flowing back into the cavity 311 through the outlet connector 318.

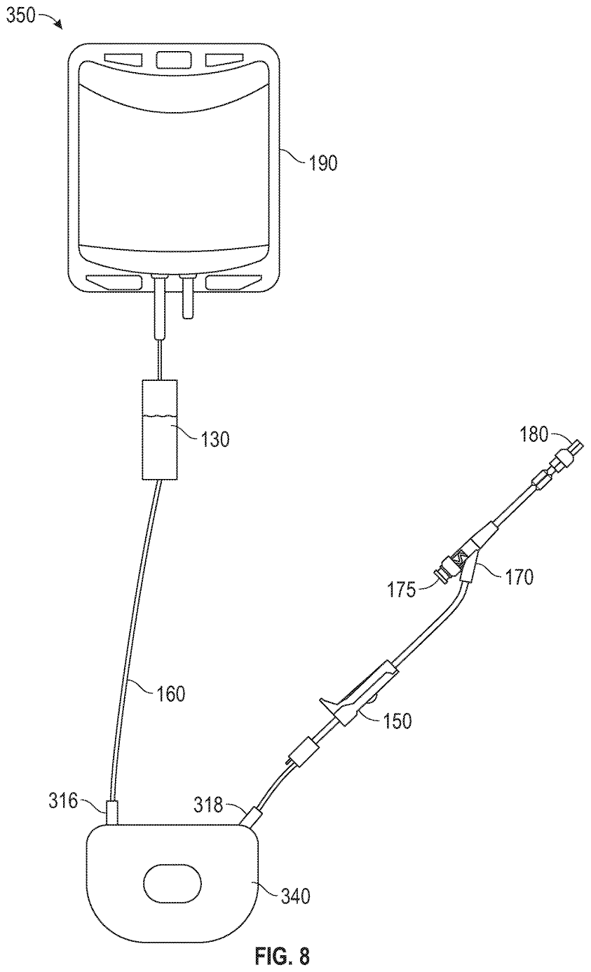

In aspects of the disclosure, the IV blood set foot pump 300 may be a reusable device. For example, an IV blood set 350 (see FIG. 8) similar to IV blood set 250 may have a disposable inner bladder 340 (e.g., similar to pump bulb 240) configured to be disposed within the cavity 311 and coupled directly to the inlet connector 316 and the outlet connector 318, while being disposed around the biasing member 330 (e.g., doughnut shaped). Thus, other than the inner bladder 340, the housing 310 and the squeezing member 320 of the IV blood set foot pump 300 are not in direct contact with the fluid (e.g., blood) flowing through the IV blood set 350, so the inner bladder 340 may be removed from the IV blood set foot pump 300 and an inner bladder 340 of a replacement IV blood set 350 inserted. Accordingly, IV blood set foot pump 300 may be used for multiple procedures or IV blood set changes between cleanings, or the IV blood set foot pump 300 may be cleaned after every use as dictated by health codes and medical device cleaning protocols.

In use, a bladder (e.g., inner bladder 340) of an IV set (e.g., IV set 350) is inserted or placed into the IV blood set foot pump 300, the inner bladder 340 being positioned between the housing 310 and the squeezing member 320. The IV set 350 is coupled to a fluid container (e.g., fluid bag 190) containing a medical fluid (e.g., blood). In an unactuated state, IV blood set foot pump 300 may not compress the inner bladder 340, thus allowing the blood to flow through the IV set 350 at a rate set by a flow controller (e.g., roller clamp 150). When quicker fluid flow is needed, the IV blood set foot pump 300 may be compressed by foot, thus squeezing the fluid out of the inner bladder 340 quickly.

In aspects of the disclosure, the IV blood set foot pump 300 may be a disposable component of an IV blood set 350a. For example, the fluid may flow directly into and out of the cavity 311 of IV blood set foot pump 300 so that the housing 310 and the squeezing member 320 come into contact with the fluid during operation of the IV blood set 350a. Here, the disposable version of the IV blood set foot pump 300 would be disposed of when the IV blood set 350a is disposed of and replaced.

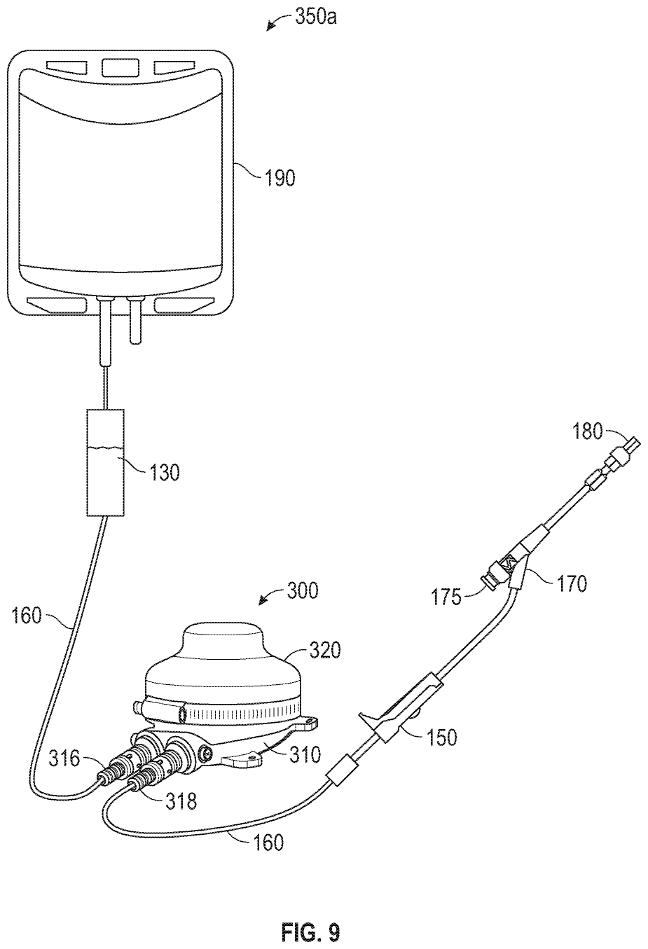

In use, an IV set 350a (see FIG. 9) similar to IV blood set 350 may have a disposable IV blood set foot pump 300 coupled to a fluid container (e.g., fluid bag 190) containing a medical fluid (e.g., blood). In an unactuated state, IV blood set foot pump 300 may not compress the squeezing member 320, thus allowing the blood to flow through the IV set 350a and through the cavity 311 of the IV blood set foot pump 300 at a rate set by a flow controller (e.g., roller clamp 150). When quicker fluid flow is needed, the IV blood set foot pump 300 may be compressed by foot, thus squeezing the fluid out of the cavity 311 quickly.

In aspects of the disclosure, the IV blood set foot pump 200, 300 may include any suitable fastener to mount the IV blood set foot pump 200, 300 to a base of an IV pole 46, a base of a bed, a floor surface and the like. For example, the fastener may be screws, rivets, a bracket, a hangar, a hook, Velcro ®, adhesive, and/or any other suitable fastener.

In one or more embodiments of the disclosure, a foot operated pumping mechanism may include a reusable device that remains affixed on an IV pole and attaches itself to a diaphragm-based pump segment of a blood set. Here, the diaphragm-based pump segment replaces a typical hand pump (e.g., hand pump 140) and may be inserted into the reusable foot pump when an increased fluid flow is needed. This arrangement provides for a quicker and efficient blood infusion and keeps the clinician's hands free for other procedures.

The reusable foot pump remains attached to the IV pole and may consist of a spring-loaded foot pedal, a fixed plate that gets attached to the IV pole and a moving plate that is pneumatically connected with the foot pedal. The fixed plate may have spring loaded guides, which along with the moving plate, provides for quick refill of the blood set pump segment. The diaphragm pump segment may have a diaphragm that gets attached to the moving plate. With pressing movement of the moving plate, the diaphragm gets pressed and pumps blood out of a chamber of the pump segment. With pulling movement of the moving plate, the diaphragm gets pulled and refills the chamber. The pump segment may also have ball valves to prevent any retrograde flow.

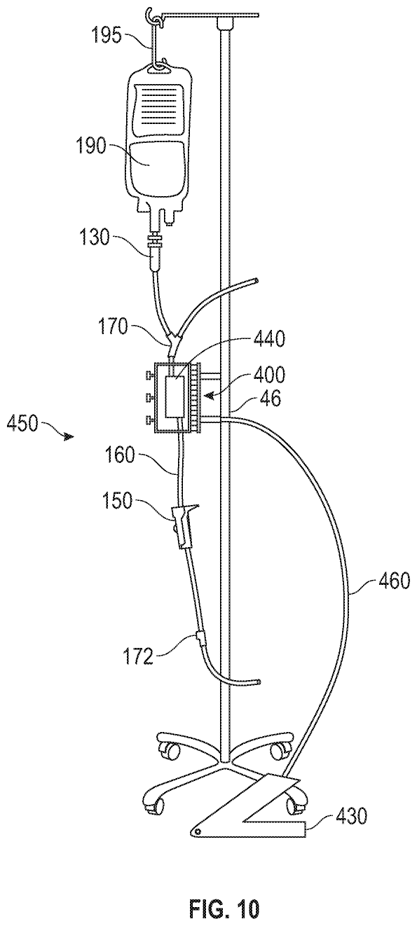

As shown in FIG. 10, an IV set 450 (e.g., IV blood set) and an IV set foot pump 400 may be coupled to a fluid source 190 (e.g., blood bag) hanging from an extension hook 195 of an IV pole 46. For example, the IV set 450 may include a drip chamber 130, a Y-junction 170, a diaphragm pump segment 440, IV tubing 160, a roller clamp 150, and a port 172. The IV set 450 may be connected downstream to a catheter that is placed into a vein of a patient as described above. In aspects of the disclosure, the IV set 450 and the IV set foot pump 400 may be used for infusion of blood. In aspects of the disclosure, the IV set 450 and the IV set foot pump 400 may be used for delivery of any suitable fluid (e.g., saline, drugs).

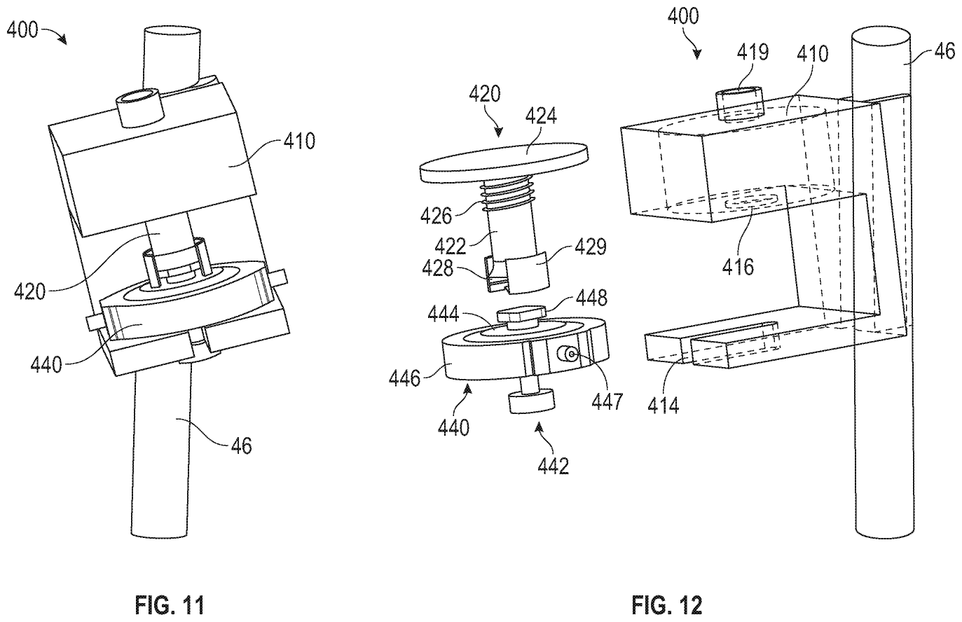

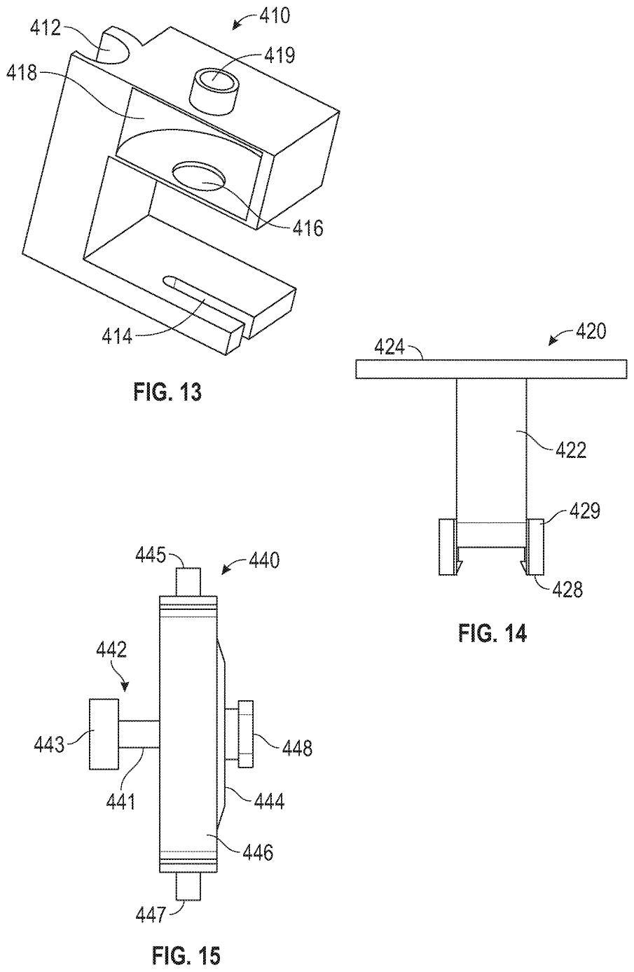

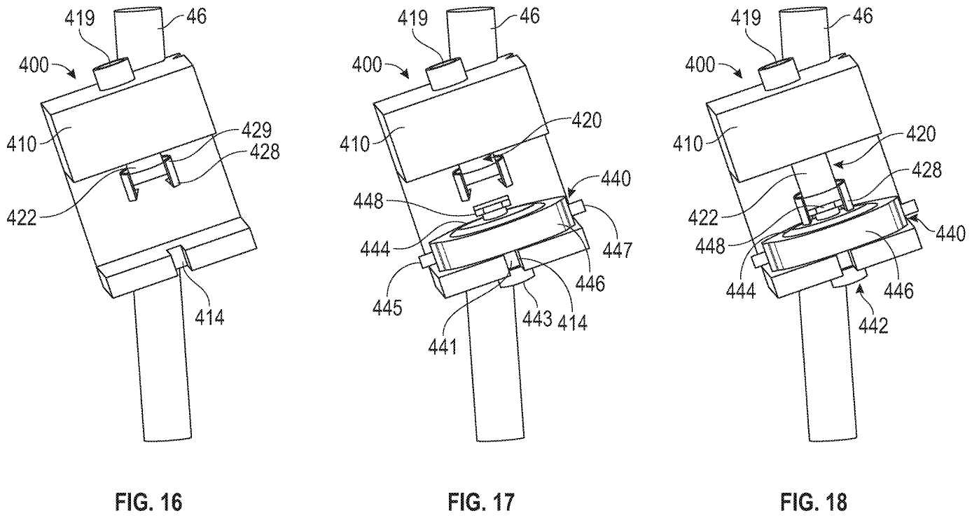

As shown in FIGS. 11-18, the IV set foot pump 400 may include a frame 410 that is configured to be fixed or coupled to the IV pole 46. The frame 410 may include an IV pole coupling channel 412 for fixing the frame 410 on the IV pole 46 (e.g., snapping the coupling channel 412 onto the IV pole 46). Here, the frame 410 may be uncoupled from the IV pole 46 by pulling the frame 410 to unsnap the coupling channel 412 from the IV pole 46. The frame 410 may also include a pump slot 414 configured to receive and hold a pump grip 442 of the diaphragm pump segment 440. For example, the pump grip 442 may have an extension member 441 that may be slidably received in the pump slot 414, as well as a retention member 443 that prevents the extension member 441 from pulling through the pump slot 414.

The frame 410 may include a piston slot 416 configured to slidably receive a shaft 422 of a piston 420 and a frame cylinder 418 configured to receive a head 424 of the piston 420. Here, the frame cylinder 418 may be a compartment within the frame 410 that is connected to both an air inlet port 419 and the piston slot 416. For example, as the head 424 moves up and down within the frame cylinder 418, the shaft 422 slides correspondingly through the piston slot 416. The air inlet port 419 of the frame 410 is configured to couple with a foot pump 430 via an air hose 460. Thus, a compression or activation of the foot pump 430 forces air through the air hose 460 and into the frame cylinder 418 via the air inlet port 419. The pressure of the air against the head 424 of the piston 420 forces the head 424 away from the air inlet port 419, causing the shaft 422 of the piston 420 to slide through the piston slot 416 and further into the frame 410. In reverse, a release of the foot pump 430 causes a suction force that pulls air back from the frame cylinder 418 through the air inlet port 419. The suction force, along with a biasing force from a spring 426 coupled or mounted on the shaft 422, causes the shaft 422 to slide in reverse through the piston slot 416 to move the head 424 towards the air inlet port 419 within the frame cylinder 418.

The diaphragm pump segment 440 may include a diaphragm 444 disposed on a chamber 446, where the diaphragm 444 is configured to be pulled (e.g., expanded) from the chamber 446 and pushed (e.g., contracted) towards the chamber 446 by one or more piston snaps disposed on the shaft 422 that are engaged with a pump snap 448 extending from the diaphragm 444, based on the slidable movement of the shaft 422. A snap release 429 for each piston snap 428 may be coupled to the shaft 422 and/or to the corresponding piston snap 428. The diaphragm pump segment 440 also includes a fluid inlet 445 and a fluid outlet 447 each disposed on the chamber 446, such as on opposing ends of the chamber 446, for example. The fluid inlet 445 and fluid outlet 447 may be coupled with IV tubing 160 of the IV set 450.

In use, when pumping of the IV set foot pump 400 in required or desired, the diaphragm pump segment 440 is placed in the frame 410 such that the extension member 441 of the pump grip 442 is correctly fit within the pump slot 414 provided in the frame 410 and the retention member 443 and the chamber 446 are disposed on opposing sides of the pump slot 414. This ensures that the diaphragm pump segment 440 stays in place during the pumping action. The pump snap 448 may be an extruded feature overmolded on the diaphragm 444, where the pump snap 448 is provided to snap lock the piston 420 to the diaphragm 444 using the piston snap 428. When the diaphragm pump segment 440 is to be removed from the frame 410, the snap release 429, which may be provided as extrusions of the piston 420, may be squeezed to cause the piston snap 428 to disengage from the pump snap 448 and allow the diaphragm pump segment 440 to be pulled out of the frame 410.

During pumping operation of the diaphragm pump segment 440, the air from the foot pump 430 enters the frame cylinder 418 through the air inlet port 419 and consequently exerts a force on the head 424 (e.g., top surface of the piston 420). This causes the shaft 422 of the piston 420 to move through the piston slot 416 towards the diaphragm pump segment 440, which in turn compresses the diaphragm 444. This action pushes fluid out of the chamber 446 through the fluid outlet 447 to the IV set 450 disposed downstream of the diaphragm pump segment 440. As the force on the foot pump 430 is released, a suction force is created that combines with the biasing force of the spring 426 results in an outward motion of the piston 420 away from the diaphragm pump segment 440, which in turn pulls the diaphragm 444 outwards from the chamber 446. This results in fluid being suctioned into the chamber 446 in through the fluid inlet 445 from the fluid source 190 via the IV set 450 disposed upstream of the diaphragm pump segment 440.

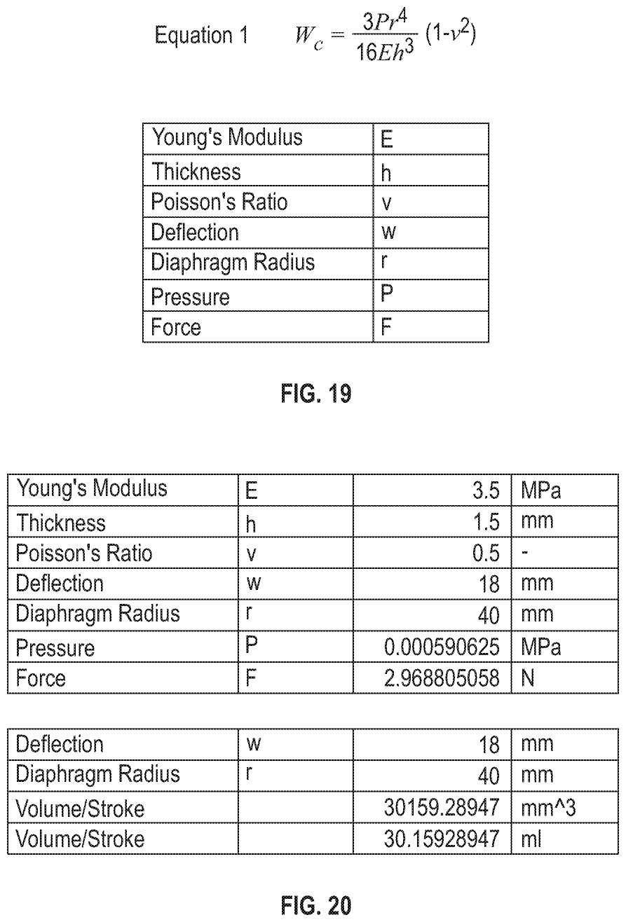

As shown in FIG. 19, an equation 1 may be used for calculation of pressure and force required to deflect the diaphragm 444 by a certain distance. For example, the diaphragm 444 may be formed of a rubber or other elastic material for which the Young Modulus is 3.5 Mpa, the thickness of the diaphragm is 1.5 mm, Poisson's ratio is 0.5, the diaphragm reflection is 18 mm and the diameter is 80 mm, the force required to deflect the diaphragm 444 is 2.968 N and the volume of fluid per stroke is 30 ml/stroke, as shown in FIG. 20.

In one or more embodiments, an IV blood set foot pump comprises: a base plate; a squeezing plate movably coupled to the base plate by a pivot member; and a biasing member coupled to the base plate and the squeezing plate, the biasing member configured to exert a biasing force against the squeezing plate when the biasing member is in a compressed state, wherein one of the base plate and the squeezing plate comprises a cavity configured to receive an IV pump bulb.

In aspects of the disclosure, the cavity is configured to receive a standard IV hand pump bulb. In aspects of the disclosure, the pivot member is a hinge pin. In aspects of the disclosure, the pivot member is a flexible hinge integrally formed with the base plate and the squeezing plate. In aspects of the disclosure, the biasing member is a compression spring.

In aspects of the disclosure, an IV blood set comprises: a pump bulb; a first IV tube coupled to an inlet of the pump bulb; a second IV tube coupled to an outlet of the pump bulb; and an IV blood set foot pump comprising: a base plate; a squeezing plate movably coupled to the base plate by a pivot member; and a biasing member coupled to the base plate and the squeezing plate, the biasing member configured to exert a biasing force against the squeezing plate when the biasing member is in a compressed state, wherein one of the base plate and the squeezing plate comprises a cavity configured to receive an IV pump bulb.

In aspects of the disclosure, a method of operating an IV blood set foot pump, comprises: coupling an IV set to a fluid source; inserting a pump bulb of the IV set into an IV blood set foot pump comprising: a base plate; a squeezing plate movably coupled to the base plate by a pivot member; and a biasing member coupled to the base plate and the squeezing plate, the biasing member configured to exert a biasing force against the squeezing plate when the biasing member is in a compressed state, wherein one of the base plate and the squeezing plate comprises a cavity configured to receive the IV pump bulb; pressing on the squeezing plate with a foot to compress the squeezing plate towards the base plate; releasing pressure on the squeezing plate with the foot to allow the squeezing plate to move back to a default position based on the biasing force from the biasing member; and repeating the pressing and releasing steps to pump the fluid from the fluid source through the IV set at a fluid flow rate that exceeds the maximum open flow fluid flow rate of the IV set itself.

In one or more embodiments, an IV blood set foot pump comprises: a housing comprising: a fluid cavity; an inlet port; and an outlet port; a squeezing member coupled to the housing; and a biasing member coupled to the housing and the squeezing member, the biasing member configured to exert a biasing force against the squeezing member when the biasing member is in a compressed state, wherein the inlet port is configured to be coupled to a first IV tube of an IV set and the outlet port is configured to be coupled to a second IV tube of the IV set.

In aspects of the disclosure, an inlet connector is coupled to the inlet port; and a check valve is disposed within the inlet connector, wherein the check valve is configured to allow fluid flow into the housing through the inlet port and to prevent fluid flow out of the housing through the inlet port. In aspects of the disclosure, an outlet connector is coupled to the outlet port; and a check valve is disposed within the outlet connector, wherein the check valve is configured to allow fluid flow out of the housing through the outlet port and to prevent fluid flow into the housing through the outlet port. In aspects of the disclosure, a securing member is coupled to the squeezing member, the securing member configured to secure the squeezing member to the housing.

In aspects of the disclosure, the squeezing member is a flexible diaphragm and the biasing member is a spring. In aspects of the disclosure, an inner bladder is disposed within the cavity and around the biasing member, the inner bladder coupled directly to inlet and outlet connectors coupled to the inlet and outlet ports, respectively, wherein the inner bladder, the inlet connector and the outlet connector are configured to be removably disposed and the rest of the IV blood set foot pump is configured to be reusable and to receive a new inner bladder, inlet connector and outlet connector.

In aspects of the disclosure, a method of operating an IV blood set foot pump comprises: coupling an IV set to a fluid source; flowing fluid from the fluid source into an IV blood set foot pump comprising: a housing comprising: a fluid cavity; an inlet port; and an outlet port; a squeezing member coupled to the housing; and a biasing member coupled to the housing and the squeezing member, the biasing member configured to exert a biasing force against the squeezing member when the biasing member is in a compressed state, wherein the inlet port is coupled to a first IV tube of the IV set and the outlet port is coupled to a second IV tube of the IV set; pressing on the squeezing member with a foot to compress the squeezing member towards the housing to increase pressure within the cavity to force the fluid quickly out of the outlet port; releasing pressure on the squeezing member with the foot to allow the squeezing member to move to return back to a default position based on the biasing force from the biasing member, the return movement of the squeezing member causing a suction pressure inward towards the cavity to force fluid upstream of the IV blood set foot pump to flow quickly into the inlet port; and repeating the pressing and releasing steps to pump the fluid from the fluid source through the IV set at a fluid flow rate that exceeds the maximum open flow fluid flow rate of the IV set itself.

In one or more embodiments, an IV blood set foot pump comprises: a frame configured to be removably coupled to an IV pole, the frame comprising: an air inlet port; a piston slot; and a pump slot configured to receive a diaphragm pump segment of an IV set; a piston moveably coupled to the frame through the piston slot; an air hose, wherein a first end of the air hose is coupled to the air inlet port of the frame; and a foot pump coupled to a second end of the air hose.

In aspects of the disclosure, the frame comprises a frame cylinder connected to the air inlet port and to the piston slot. In aspects of the disclosure, the piston comprises: a shaft having a first end and a second end, the shaft slidably disposed within the piston slot; a head at the first end of the shaft, the head movably disposed within the frame cylinder; a piston snap at the second end of the shaft, the piston snap configured to engage with a pump snap of the diaphragm pump segment of the IV set; and a snap release at the second end of the shaft, the snap release configured to release the engagement of the piston snap with the pump snap. In aspects of the disclosure, the frame is configured to receive the diaphragm pump segment with an extension member of a pump grip disposed within the pump slot, a retention member of the pump grip disposed on an exterior side of the pump slot, and a chamber of the diaphragm pump segment disposed on an interior side of the pump slot.

In aspects of the disclosure, a method of operating an IV blood set foot pump comprises: coupling an IV set to a fluid source; inserting a diaphragm pump segment of the IV set into the frame of an IV blood set foot pump comprising: a frame configured to be removably coupled to an IV pole, the frame comprising: an air inlet port; a piston slot; and a pump slot configured to receive the diaphragm pump segment of the IV set; a piston moveably coupled to the frame through the piston slot; an air hose, wherein a first end of the air hose is coupled to the air inlet port of the frame; and a foot pump coupled to a second end of the air hose; coupling a piston snap of the piston with a pump snap of the diaphragm pump segment; pressing on the foot pump with a foot to force air into a frame cylinder of the frame through the air hose and the air inlet port of the frame, causing the piston to move inward within the frame and compress a diaphragm coupled to a chamber of the diaphragm pump segment to force fluid within the chamber out of an outlet port of the diaphragm pump segment and through the IV set downstream of the diaphragm pump segment; releasing pressure with the foot on the foot pump to allow the piston to move to return back to a default position within the frame based on at least one of a biasing force from a spring coupled to the shaft of the piston and a suction force from air being pulled out of the frame cylinder through the air inlet port and the air hose into the foot pump, the return movement of the piston causing the diaphragm to pull outward from the chamber and cause a suction pressure inward towards the chamber to force fluid upstream of the diaphragm pump segment to flow quickly into the chamber through an inlet port of the diaphragm pump segment; and repeating the pressing and releasing steps on the foot pump to pump the fluid from the fluid source through the IV set at a fluid flow rate that exceeds the maximum open flow fluid flow rate of the IV set itself.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) blood set foot pump comprising:
a housing comprising:
a fluid cavity;
an inlet port; and
an outlet port;
a squeezing member coupled to the housing; and
a biasing member coupled to the housing and the squeezing member, the biasing member configured to exert a biasing force against the squeezing member when the biasing member is in a compressed state,
wherein the inlet port is configured to be coupled to a first IV tube of an IV set and the outlet port is configured to be coupled to a second IV tube of the IV set.

2. The IV blood set foot pump of claim 1, further comprising:
an inlet connector coupled to the inlet port; and
a check valve disposed within the inlet connector,
wherein the check valve is configured to allow fluid flow into the housing through the inlet port and to prevent fluid flow out of the housing through the inlet port.

3. The IV blood set foot pump of claim 1, further comprising:
an outlet connector coupled to the outlet port; and
a check valve disposed within the outlet connector,
wherein the check valve is configured to allow fluid flow out of the housing through the outlet port and to prevent fluid flow into the housing through the outlet port.

4. The IV blood set foot pump of claim 1, further comprising:
a securing member coupled to the squeezing member, the securing member configured to secure the squeezing member to the housing.

5. The IV blood set foot pump of claim 1, wherein the squeezing member is a flexible diaphragm and the biasing member is a spring.

6. The IV blood set foot pump of claim 1, further comprising:
an inner bladder disposed within the cavity and around the biasing member, the inner bladder coupled directly to inlet and outlet connectors coupled to the inlet and outlet ports, respectively.

7. The IV blood set foot pump of claim 6, wherein the inner bladder, the inlet connector and the outlet connector are configured to be removably disposed.

8. The IV blood set foot pump of claim 7, wherein the rest of the IV blood set foot pump is configured to be reusable and to receive a new inner bladder, inlet connector and outlet connector.

9. A method of operating the IV blood set foot pump of claim 1, the method comprising:
coupling an IV set to a fluid source;
flowing fluid from the fluid source into the IV blood set foot pump;
pressing on the squeezing member with a foot to compress the squeezing member towards the housing to increase pressure within the cavity to force the fluid quickly out of the outlet port;
releasing pressure on the squeezing member with the foot to allow the squeezing member to move to return back to a default position based on the biasing force from the biasing member, the return movement of the squeezing member causing a suction pressure inward towards the cavity to force fluid upstream of the IV blood set foot pump to flow quickly into the inlet port; and
repeating the pressing and releasing steps to pump the fluid from the fluid source through the IV set at a fluid flow rate that exceeds the maximum open flow fluid flow rate of the IV set itself.

10. An intravenous (IV) blood set foot pump comprising:
a frame configured to be removably coupled to an IV pole, the frame comprising:
an air inlet port;
a piston slot; and
a pump slot configured to receive a diaphragm pump segment of an IV set;
a piston moveably coupled to the frame through the piston slot;
an air hose, wherein a first end of the air hose is coupled to the air inlet port of the frame; and
a foot pump coupled to a second end of the air hose.

11. The IV blood set foot pump of claim 10, wherein the frame comprises a frame cylinder connected to the air inlet port and to the piston slot.

12. The IV blood set foot pump of claim 11, wherein the air inlet port is disposed on a top surface of the frame and the piston slot is disposed in a bottom surface of the frame cylinder.

13. The IV blood set foot pump of claim 12, wherein the frame cylinder comprises a compartment disposed below the top surface of the frame and connected to the air inlet port.

14. The IV blood set foot pump of claim 11, wherein the piston comprises:
a shaft having a first end and a second end, the shaft slidably disposed within the piston slot; and
a head at the first end of the shaft, the head movably disposed within the frame cylinder.

15. The IV blood set foot pump of claim 14, wherein the piston further comprises:
a piston snap at the second end of the shaft, the piston snap configured to engage with a pump snap of the diaphragm pump segment of the IV set; and
a snap release at the second end of the shaft, the snap release configured to release the engagement of the piston snap with the pump snap.

16. The IV blood set foot pump of claim 15, wherein the frame is configured to receive the diaphragm pump segment with an extension member of a pump grip disposed within the pump slot, a retention member of the pump grip disposed on an exterior side of the pump slot, and a chamber of the diaphragm pump segment disposed on an interior side of the pump slot.

17. The IV blood set foot pump of claim 11, wherein the pump slot is disposed in a bottom surface of the frame and comprises an opening in a front surface of the frame, wherein a space between the frame cylinder and the bottom surface of the frame is configured to receive the diaphragm pump segment.

18. The IV blood set foot pump of claim 10, wherein the frame comprises:
an IV pole coupling channel disposed on a rear end of the frame and configured to snap fit the frame to the IV pole.

19. A method of operating the IV blood set foot pump of claim 10, the method comprising:
coupling an IV set to a fluid source;
inserting a diaphragm pump segment of the IV set into the frame of the IV blood set foot pump;
coupling the piston snap of the piston with a pump snap of the diaphragm pump segment;
pressing on the foot pump with a foot to force air into a frame cylinder through the air hose and the air inlet port of the frame, causing the piston to move inward within the frame and compress a diaphragm coupled to a chamber of the diaphragm pump segment to force fluid within the chamber out of an outlet port of the diaphragm pump segment and through the IV set downstream of the diaphragm pump segment;
releasing pressure with the foot on the foot pump to allow the piston to move to return back to a default position within the frame based on at least one of a biasing force from a spring coupled to a shaft of the piston and a suction force from air being pulled out of the frame cylinder through the air inlet port and the air hose into the foot pump, the return movement of the piston causing the diaphragm to pull outward from the chamber and cause a suction pressure inward towards the chamber to force fluid upstream of the diaphragm pump segment to flow quickly into the chamber through an inlet port of the diaphragm pump segment; and
repeating the pressing and releasing steps on the foot pump to pump the fluid from the fluid source through the IV set at a fluid flow rate that exceeds the maximum open flow fluid flow rate of the IV set itself.

20. An IV blood set comprising:
the IV blood set foot pump of claim 10;
a diaphragm pump segment comprising:
a chamber;
a diaphragm coupled to a first side of the chamber;
a pump snap coupled to the diaphragm;
a pump grip coupled to a second side of the chamber;
an inlet port; and
an outlet port;
a first IV tube coupled to the inlet port of the diaphragm pump segment; and
a second IV tube coupled to an outlet port of the diaphragm pump segment.
